(19) United States
(12) Patent Application Publication
(10) Pub. No.: US 2009/0131660 A1
(43) Pub. Date: May 21, 2009

(54) SYNTHESIS OF 3-[4-(1,1-DIMETHYL-PROPYL)-PHENYL]-2-METHYL-PROPIONALDEHYDE AND cis-4--2,6-DIMETHYL-MORPHOLINE (AMOROLFINE)

(75) Inventors: JEAN-GUY BOITEAU, SAINT-AUNES (FR); BRANISLAV MUSICKI, NICE (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/244,788

(22) Filed: Oct. 3, 2008

(30) Foreign Application Priority Data
Apr. 3, 2006 (EP) 06290532.8
Mar. 29, 2007 (EP) PCT/EP2007/053050

(51) Int. Cl. C07D 265/30; C07C 45/27; C07C 17/013

(52) U.S. Cl. 544/178; 568/437; 570/206

(57) ABSTRACT

3-[4-(1,1-Dimethyl-propyl)-phenyl]-2-methyl-propionaldehyde and cis-4-{3-[4-(1,1-dimethyl-propyl)-phenyl]-2-methyl-propyl}-2,6-dimethyl-morpholine (Amorolfine) are synthesized, first by Heck reacting a compound of general formula (VI):

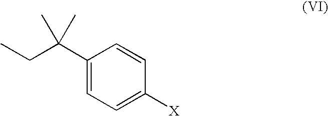

with 2-methyl-prop-2-en-1-ol of formula (VII):

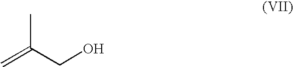

in the presence of a palladium catalyst and a base, in a solvent selected from among N,N-dimethylformamide, polar protic and non-polar organic solvents, and at a temperature ranging from 60° C. to 150° C., and then conducting an amino reduction by reacting the 3-[4-(1,1-dimethyl-propyl)-phenyl]-2-methyl-propionaldehyde of formula (II) with cis-2,6-dimethyl morpholine of formula (IV):

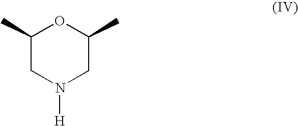

in the presence of a reducing agent and in a solvent.

### CROSS-REFERENCE TO PRIORITY/PCT APPLICATIONS

This application claims priority under 35 U.S.C. § 119 of EP 06290532.8, filed Apr. 3, 2006, and is a continuation/national phase of PCT/EP 2007/053050, filed Mar. 29, 2007, and designating the United States (published in the English language on Oct. 11, 2007 as WO 2007/113218 A1), each hereby expressly incorporated by reference in its entirety and each assigned to the assignee hereof.

### BACKGROUND OF THE INVENTION

1. Technical Field of the Invention

The present invention relates to a novel process for the synthesis of 3-[4-(1,1-dimethyl-propyl)-phenyl]-2-methyl-propionaldehyde and cis-4-{3-[4-(1,1-dimethyl-propyl)-phenyl]-2-methyl-propyl}-2,6-dimethyl-morpholine or salt thereof.

2. Description of Background and/or Related and/or Prior Art

Amorolfine, cis-4-{3-[4-(1,1-dimethyl-propyl)-phenyl]-2-methyl-propyl-2,6-dimethyl-morpholine of formula (I) is a potent antifungal drug exhibiting a wide spectrum of in vitro activity. It belongs to a new chemical class of anti-mycotics and exhibits a broad spectrum of antifungal activity against dermatophytes, dimorphic fungi, Candida albicans, Cryptococus neoformans and certain dematiaceae. It finds a major application as the active ingredient in nail lacquer as a topical antifungal in the treatment of onychomycosis and as atopical anti-mycotic indicated for the treatment of dermatomycosis.

Zhixiang describes a multi-step synthesis of Amorolfine from tert-pentylbenzene (F. Zhixiang & coil., Zhongguo Yaowu Huaxue Zazhi, 10(1), 64-65, (2000)). By hydroxymethylation and bromination from tert-pentylbenzene, 4-bromomethyl-tert-pentylbenzene is prepared. It is coupled with methyl malonic ester, hydrolyzed and decarboxylated to afford 2-methyl-3-(4-tert-pentylphenyl) propionic acid. This intermediate is amidated with cis-2,6-dimethylmorpholine and reduced to provide Amorolfine of formula (I).

Hoffmann-La Roche patent (FR 2,463,767 or EP 24,334) describes other synthetic pathways for the production of Amorolfine of formula (I) or its hydrochloride salt of formula (Ia). In the Hoffmann-La Roche patent, Amorolfine is produced as a racemic mixture of the cis isomers by three different synthetic pathways.

A first synthetic pathway is a nucleophilic substitution on 1-(3-halo-2-methyl-propyl)-4-(1,1-dimethyl-propyl)-benzene (halo=chlorine, bromine or iodine) by cis-2,6-dimethyl morpholine to produce Amorolfine of formula (I)

A second synthetic pathway is a reduction of a compound of the formula:

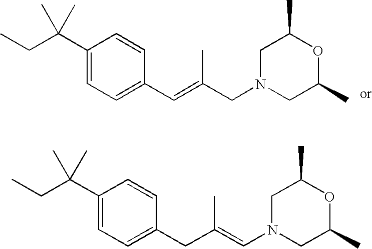

to produce Amorolfine of formula (I).

A third synthetic pathway is an amino reduction from (E)-2-Methyl-3-phenyl-propenal and cis-2,6-dimethyl morpholine to produce cis-2,6-dimethyl-4-(2-methyl-3-phenyl-propyl)-morpholine followed by a Friedel-Craft reaction with 2-methyl-2-butanol to produce Amorolfine of formula (I).

Also, Fenpropimorph, 4-[3-(4-tert-butylphenyl)-2-methylpropyl]-2,6-dimethylmorpholine, which is a pesticide, specifically categorized as a morpholine fungicide, has recently been synthesized by Forsyth in a two-step one pot process (S. A. Forsyth & col; Organic Process Research & Development, 10, 94-102, (2006)).

This process includes a Heck reaction from 4-tert-butyl-iodobenzene and 2-methyl-prop-2-en-1-ol catalyzed by palladium chloride (PdCl₂) in ionic liquid solvent followed by an amino reduction in the presence of cis-2,6-dimethyl morpholine catalyzed by palladium on carbon (Pd/C) under hydrogen pressure in ionic liquid solvent.

### SUMMARY OF THE INVENTION

It has now been found that Amorolfine or its salts can be prepared in a two-step one pot process entailing a Heck reaction from (1,1-dimethyl-propyl)-4-iodo-benzene and 2-methyl-prop-2-en-1-ol, catalyzed by palladium catalyst such as palladium acetate (PdOAc₂) in N,N-dimethylformamide (DMF) followed by an amino reduction in alcohol in the presence of cis-2,6-dimethyl morpholine and a reducing agent such as hydrogen gas/palladium catalyst or mixed metal hydrides, in alcoholic solvents.

This novel process is unique for the synthesis of Amorolfine.

The solvent employed according to the invention for the Heck reaction is DMF, polar protic or non-polar organic solvents.

Likewise, the solvent employed according to the invention for the amino reduction reaction is alcohol.

With such a two-step one pot process, the production of Amorolfine is optimized.

Thus, the present invention features a novel process for preparing 3-[4-(1,1-dimethyl-propyl)-phenyl]-2-methyl-propionaldehyde of formula (II):

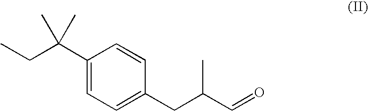

comprising Heck reacting a compound of general formula (VI):

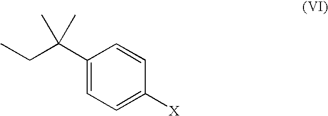

in which X is a halide such as bromine, chlorine, iodine or fluorine, or a trifluoromethane sulfonate radical (OSO₂CF₃) with 2-methyl-prop-2-en-1-ol of formula (VII):

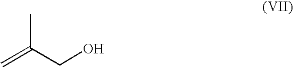

in the presence of a palladium catalyst and a base in a solvent selected from among N,N-dimethylformamide (DMF), polar protic and non-polar organic solvents, at temperature ranging from 60° C. to 150° C.

This invention also features a novel process for the synthesis of Amorolfine of formula (I):

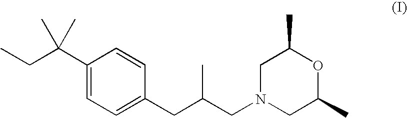

or salt thereof, comprising, in a first step, Heck coupling a compound of general formula (VI):

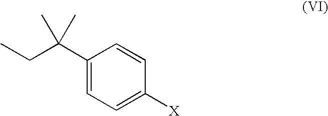

in which X is a halide such as bromine, chlorine, iodine or fluorine or a trifluoromethane sulfonate radical (OSO₂CF₃) with 2-methyl-prop-2-en-1-ol of formula (VII):

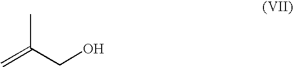

in the presence of a palladium catalyst and a base in a solvent selected from among N,N-dimethylformamide (DMF), polar protic and non-polar organic solvents, at a temperature ranging from 60° C. and 150° C., to provide 3-[4-(1,1-dimethyl-propyl)-phenyl]-2-methyl-propionaldehyde of formula (II):

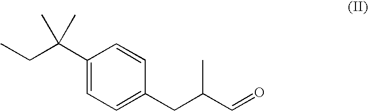

and, in a second step (amino reduction step), reacting 3-[4-(1,1-dimethyl-propyl)-phenyl]-2-methyl-propionaldehyde of formula (II) with cis-2,6-dimethyl morpholine of formula (IV):

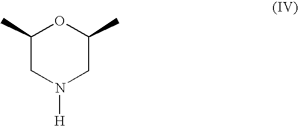

in the presence of a reducing agent in a solvent to produce cis-4-{3-[4-(1,1-dimethyl-propyl)-phenyl]-2-methyl-propyl}-2,6-dimethyl-morpholine of formula (I) (Amorolfine):

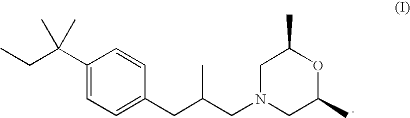

This compound can be further converted to a corresponding salt of cis-4-{3-[4-(1,1-dimethyl-propyl)-phenyl]-2-methyl-propyl}-2,6-dimethyl-morpholine. The salification can be carried out by the addition of an acid, such as hydrochloric acid.

### DETAILED DESCRIPTION OF BEST MODE AND SPECIFIC/PREFERRED EMBODIMENTS OF THE INVENTION

The two steps of the present invention as described above can be advantageously carried out in a “one pot” process without isolation of the aldehyde intermediate of formula (II).

The preferred salt of cis-4-{3-[4-(1,1-dimethyl-propyl)-phenyl]-2-methyl-propyl}-2,6-dimethyl-morpholine is the hydrochloride salt of formula (Ia):

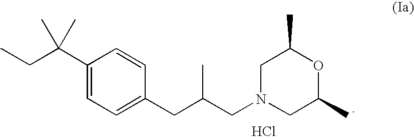

Particularly, the palladium catalyst employed in the Heck reaction conditions of the invention is selected from among palladium(II)acetate, palladium(II)chloride, tetrakis(triphenylphosphine)-palladium(0), palladium on activated carbon, dichloro[1,1′-bis(diphenylphosphino)ferrocene]palladium(II), dichloro-bis-triphenylphosphino palladium(II).

Notably, a phosphinic ligand can be employed in combination with the palladium catalyst. Commonly phosphinic ligands employed are, for example, triphenylphosphine, tri-o-tolylphosphine, tri-m-tolylphosphine or tri-p-tolylphosphine.

In this first step, the organic solvents employed according to the invention are N,N-dimethylformamide (DMF), polar protic solvent as for example methanol, ethanol, n-propanol, i-propanol, n-butanol, i-butanol, t-butanol or appropriate mixture of these solvents with water or non-polar solvent as for example tetrahydrofuran, ethyl acetate, toluene, o-xylene, m-xylene, p-xylene.

The reaction is carried out in the presence of a base such as a tertiary amine, as for example triethylamine, tripropylamine, tributylamine, diisopropylethylamine, a metal carbonate, as for example sodium carbonate, sodium bicarbonate, potassium carbonate, potassium bicarbonate or a metal acetate as for example potassium acetate or sodium acetate.

Advantageously, the reaction is carried out in an inert atmosphere, such as under nitrogen or argon gas in a suitable reaction vessel.

The temperatures are maintained from about 60° C. to about 150° C. for about 30 min to 24 h.

Advantageously, the molar ratio of the two starting materials (VI)/(VII) range from (VI)/(VII)=1/1 to (VI)/(VII)=1/2.

According to the invention, the preferred conditions for the Heck reaction are DMF as solvent, an homogeneous palladium catalyst such as palladium(II) acetate, a base such as sodium carbonate, a reaction time of from 8 hours to 10 hours, for example 9 hours, at a temperature ranging from 80° C. to 110° C.

As starting material, the preferred compound of general formula (VI) is the (1,1-dimethyl-propyl)-4-iodo-benzene of formula (VIa):

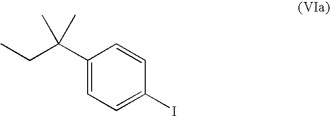

Only one synthetic method is reported in the literature for this starting material. Unfortunately, the conditions (HI—I₂) employed gave only a moderate 60% yield starting from (1,1-dimethyl-propyl)-benzene of formula (VIII) (E. Boedtker, Bull. Soc. Chim., 45, 645-650, (1929)).

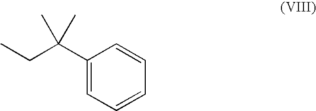

A novel process for an iodination reaction of a compound of formula (VIII) is achieved by employing sodium metaperiodate and iodine in a mixture of acetic acid and acetic anhydride, followed by the addition of sulfuric acid. Under these conditions, (1,1-dimethyl-propyl)-4-iodo-benzene of formula (VIa) is obtained in excellent yield (98%).

The amino reduction step is performed with a reducing agent such as:

hydrogen gas in the presence of a palladium catalyst, such as, for example, palladium on activated carbon, palladium on activated carbon in the presence of metal salts such as Ba(OH)₂, Ca(OH)₂, CaCO₃, BaCO₃ or Perlmans catalyst Pd(OH)₂.

or mixed metal hydrides such as metal borohydride, for example sodium borohydride (NaBH₄) and lithium borohydride (LiBH₄), or a metal cyano borohydride, such as, for example, sodium cyano borohydride (NaCNBH₃) and lithium cyanoborohydride (LiCNBH₃).

The solvent in which the amino reduction is carried out is N,N-dimethylformamide, a polar protic solvent such as methanol, ethanol, propanol, i-propanol, butanol, iso-butanol, tert-butanol or a non-polar organic solvent such as toluene, tetrahydrofurane, ethyl acetate.

The temperature for this amino reduction may typically be established at no more than 45° C., preferably from 20° C. and 45° C., and more preferably from 20° C. and 30° C. in the case of metal catalyst such as palladium catalyst, and preferably from 0° C. and 30° C. in the case of a mixed metal hydride.

When the two steps of the present invention as described above are carried out advantageously in a “one pot” process without isolation of the aldehyde intermediate of formula (II), the preferred conditions are to:

a) first react (1,1-dimethyl-propyl)-4-iodo-benzene of formula (VIa) with 2-methyl-prop-2-en-1-ol of formula (VII) in DMF as solvent and in the presence of palladium acetate (PdOAc₂) as catalyst and sodium bicarbonate (NaHCO₃) as base, at 100° C. during 9 h;

b) filtrate the catalyst through Celite, then add ethanol, acetic acid and cis-2,6-dimethylmorpholine of formula (IV);

c) cool the mixture to −5° C., then add sodium borohydride (NaBH₄);

d) after work-up, dissolve the crude product obtained from step c) in diisopropyl oxide (iPr₂O) and add a solution of hydrochloric acid gas (HCl) in ethyl acetate (EtOAc);

e) isolate cis-4-{3-[4-(1,1-dimethyl-propyl)-phenyl]-2-methyl-propyl}-2,6-dimethyl-morpholine hydrochloride of formula (Ia) obtained by filtration.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Two-Step Synthesis of cis-4-{3-[4-(1,1-dimethyl-propyl)-phenyl]-2-methyl-propyl}-2,6-dimethyl-morpholine Hydrochloride (Ia) Employing NaBH₄ as Reducing Agent

First Step:

### Preparation of (1,1-dimethyl-propyl)-4-iodo-benzene (Via)

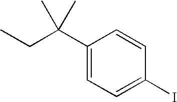

To a mixture of AcOH (1188 mL) and Ac₂O (594 mL) was added sodium periodate (79.83 g, 0.373 mol,) and iodine (280.94 g, 1.107 mol,). Then the mixture was cooled in an ice-bath and concentrated. sulfuric acid (184 mL, 3.71 mol) was added drop-wise over 20 min so that temperature did not exceed 12° C. After the addition was complete, (1,1-dimethyl-propyl)-benzene (256.76 g, 1.732 mol, 1.0 eq)) was added at once and the stirring continued for 24 h. The reaction was then partitioned from heptane-10% EtOAc (1 L) and water (2 L). The organic phase was separated and was washed with water (2 L) containing Na₂SO₃ (50 g). Then the organic phase was dried over MgSO₄ and the solvents were evaporated.

457.87 g of (1,1-dimethyl-propyl)-4-iodo-benzene were isolated as a liquid (96% yield). This material was pure enough to be used in the next step without purification.

¹H NMR (400 MHz, CDCl₃) δ: 0.73 (3H, t, J=7.4 Hz), 1.31 (6H, s), 1.67 (2H, q, J=7.4 Hz), 7.13 (2H, d, J=8.56 Hz), 7.66 (2H, d, J=8.56 Hz);

¹³C NMR (100 MHz, CDCl₃) δ: 9.59 (CH₃), 28.78 (2CH3), 37.16 (CH2), 38.3 (C_{Al}), 91.12 (C_{Ar}), 128.72 (CH_{Ar}), 137.46 (CH_{Ar}), 149.62 (C_{Ar}).

### Preparation of 3-[4-(1,1-Dimethyl-propyl)-phenyl]-2-methyl-propionaldehyde (II)

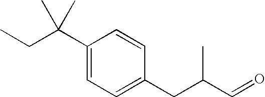

Through a mixture of (1,1-dimethyl-propyl)-4-iodo-benzene (137.07 g, 0.50 mol), palladium(II) acetat (1.122 g, 5 mmol) and sodium bicarbonate (50.40 g, 0.60 mol) in dry DMF (500 mL) was bubbled nitrogen for 10 min. Then 2-methyl-prop-2-en-1-ol (54.083 g, 0.75 mol) was added and nitrogen was bubbled for another ten minutes. The reaction mixture was heated under nitrogen for 9 h at 100° C. The reaction was cooled and was filtered through a thin layer of Celite. The Celite was then washed with DMF (300 mL). The reaction was then partitioned from H₂O (2 L) and heptane/EtOAc (9/1) (2×1 L). The combined organic layers were washed with H₂O (1 L) and were dried over MgSO₄. The solvents were evaporated.

109.63 g of 3-[4-(1,1-dimethyl-propyl)-phenyl]-2-methyl-propionaldehyde were isolated as crude product. This intermediate was purified by distillation at 112° C. under 0.06 mbar to afford 84 g of pure 3-[4-(1,1-dimethyl-propyl)-phenyl]-2-methyl-propionaldehyde (77% yield).

¹H NMR (400 MHz, CDCl₃) δ: 0.69 (3H, t, J=7.45), 1.11 (3H, d, J=6.87), 1.29 (6H, s), 1.65 (2H, q, J=7.43), 2.60 (1HAB, dd, J=8.18, 13.52 Hz), 2.69 (1H, sex, J=7.06), 3.08 (1H, dd, J=5.87, 13.54 Hz), 7.12 (2H, d, J=8.27), 7.27 (2H, d, J=8.27), 9.75 (1H, s);

¹³C NMR (100 MHz, CDCl₃) δ: 9.6 (CH₃), 13.7 (CH₃), 25.8 (CH₃), 36.6 (CH₂), 37.3 (CH₂), 38.0©, 48.5 (CH), 126.4 (CH_{Ar}), 128.6 (CH_{Ar}), 136.0 (C_{Ar}), 148.0 (C_{Ar}), 205.1 (C═O).

Second Step:

### Preparation of cis-4-{3-[4-(1,1-Dimethyl-propyl)-phenyl]-2-methyl-propyl}-2,6-dimethyl-morpholine Hydrochloride (Ia)

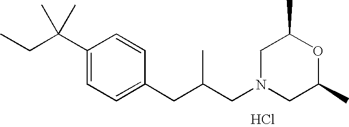

3-[4-(1,1-dimethyl-propyl)-phenyl]-2-methyl-propionaldehyde (109.63 g; 0.50 mol) obtained in the previous step were dissolved in absolute ethanol (500 mL). To the ethanol solution was added at 0° C. (ice/water) AcOH (30 mL, 0.5 mol) and cis-2,6-dimethyl-morpholine (69.108 g, 0.60 mol) and the reaction was stirred at RT for 30 min. It was then cooled to (−15° C.) and NaBH₄ (15.93 g, 0.42 mol) was added in portions during 1 h. Stirring was continued at 0° C. for 2 h and the reaction was quenched by drop-wise addition of NaOH solution in water (30 g, 0.75 mol, /100 mL H₂O) to pH=12. The reaction was then partitioned from H₂O (2 L) and hept/EtOAc (9/1) (2×1 L). The organic phases were combined, washed with the H₂O (1 L), and dried over MgSO4. After evaporation of the solvents, the residue (147.14 g) was dissolved in iPr₂O (500 mL) and cooled to 0° C. To this solution was added the solution of HCl gas in EtOAc (150 ml, ˜4M) drop-wise (30 min) and with stirring. White precipitate that was formed during HCl addition was filtered 30 min after completion of the addition. It was washed with iPr₂O (300 mL) and was dried in vacuo at 40° C. over 48 h providing 118.79 g of cis-4-{3-[4-(1,1-dimethyl-propyl)-phenyl]-2-methyl-propyl}-2,6-dimethyl-morpholine hydrochloride as white powder. (67% yield) (Melting Point=205.9° C.)

¹H NMR (400 MHz, CDCl₃) δ: 0.63 (3H, t, J=7.39 Hz), 1.12 (3H, d, J=6.30 Hz), 1.13 (3H, d, J=6.32 Hz), 1.24 (6H, s), 1.26 (3H, d, J=6.65 Hz), 1.59 (2H, q, J=7.49 Hz), 2.05 (1H, q, J=10.77 Hz), 2.26 (1H, q, J=10.80 Hz), 2.27-2.35 (1H, m), 2.56-2.66 (2H, m), 2.76-2.89 (2H, m), 3.22 (2H, t, J=13.28 Hz), 4.36-4.44 (1H, m), 4.46-4.54 (1H, m), 7.06 (2H, d, J=8.22 Hz), 7.23 (2H, d, J=8.22 Hz), 12.55 (1H, b)

### Example 2

### One Pot Synthesis of cis-4-{3-[4-(1,1-dimethyl-propyl)-phenyl]-2-methyl-propyl}-2,6-dimethyl-morpholine hydrochloride (Ia) Employing NaBH₄ as Reducing Agent

Through a mixture of (1,1-dimethyl-propyl)-4-iodo-benzene (137.07 g, 050 mol), palladium(II) acetat (1.122 g, 5 mmol) and sodium bicarbonate (50.40 g, 0.60 mol) in dry DMF (500 mL) was bubbled nitrogen for 10 min. Then 2-methyl-prop-2-en-1-ol (54.083 g, 0.75 mol) was added and nitrogen bubbled for another ten minutes. The reaction mixture was heated under nitrogen for 9 h at 100° C. The reaction was cooled (0° C.) and was filtered through a thin layer of Celite. The Celite was then washed with cold DMF (300 mL). To DMF solution was added EtOH (abs. 700 mL) followed by AcOH (30 mL) and cis-2,6-dimethylmorpholine (73.9 mL). After 15 min stirring at RT, the reaction was cooled to −5° C. and NaBH₄ (15.93 g, 0.42 mol) was added in portions over 1 h 30 min so that temperature did not exceed 2-3° C. Stirring was continued at 0° C. for 1 h and the reaction was quenched by drop-wise addition of NaOH solution in water at 0° C. (30 g, 0.75 mol, /100 mL H₂O) to pH=12. The reaction was then partitioned from H₂O (2 L) and Hept/EtOAc (9/1) (2×1 L). The organic phases were combined, washed with the H₂O (1 L), and dried over MgSO₄. After evaporation of the solvents, the residue (147.14 g) was dissolved in iPr₂O (500 mL) and cooled to 0° C. To this solution was added the solution of HCl gas in EtOAc (150 ml, ˜4M) drop-wise (30 min) and with stirring. White precipitate was formed during HCl addition and was filtered 30 min after completion of the addition. It was washed with iPr₂O (300 mL) and was dried in vacuo at 40° C. during 48 h providing 112.34 g of cis-4-{3-[4-(1,1-dimethyl-propyl)-phenyl]-2-methyl-propyl}-2,6-dimethyl-morpholine hydrochloride as white powder. (64% yield) (Melting Point=205.8° C.).

¹H NMR (400 MHz, CDCl₃) δ: 0.63 (3H, t, J=7.39 Hz), 1.12 (3H, d, J=6.30 Hz), 1.13 (3H, d, J=6.32 Hz), 1.24 (6H, s), 1.26 (3H, d, J=6.65 Hz), 1.59 (2H, q, J=7.49 Hz), 2.05 (1H, q, J=10.77 Hz), 2.26 (1H, q, J=10.80 Hz), 2.27-2.35 (1H, m), 2.56-2.66 (2H, m), 2.76-2.89 (2H, m), 3.22 (2H, t, J=13.28 Hz), 4.36-4.44 (1H, m), 4.46-4.54 (1H, m), 7.06 (2H, d, J=8.22 Hz), 7.23 (2H, d, J=8.22 Hz), 12.55 (1H, b).

### Example 3

### Two-Step Synthesis of cis-4-{3-[4-(1,1-dimethyl-propyl)-phenyl]-2-methyl-propyl}-2,6-dimethyl-morpholine Hydrochloride (Ia) Employing Hydrogen and Palladium on Carbon as Reducing Agent

First Step:

Same as Example 1.

Second Step:

3-[4-(1,1-dimethyl-propyl)-phenyl]-2-methyl-propionaldehyde (100.56 g, 0.46 mol) obtained as described in Example 1-b) was dissolved in absolute methanol (500 mL). To the methanol solution cooled at 0° C. (ice/water) was added ACOH (30 mL, 0.50 mol) and cis-2,6-dimethylmorpholine (69.108, 0.60 mol) and the reaction was stirred at room temperature for 30 min. It was then placed under nitrogen and Pd/C 10% (5.00 g) added to it. Nitrogen was replaced with hydrogen and the reaction was heated at 37° C. under hydrogen pressure. At the end of the reduction. the reaction mixture was filtered through Celite. The Celite was washed with MeOH (200 mL), then aqueous NaOH solution was added to the methanolic reaction mixture until pH=12. The reaction mixture was partitioned with Hept/EtOAc (9/1) (2×1 L), the organic phase was washed with water (1 L) and was dried over MgSO₄. After evaporation of the solvents, the residue (144.32 g) was dissolved in iPr₂O (500 mL) and cooled to 0° C. To this solution was added a solution of HCl gas in EtOAc (150 ml, ˜4M) drop-wise (30 min) and with stirring. White precipitate formed during HCl in EtOAc addition was filtered 30 min after completion of the addition. It was washed with iPr₂O (300 mL) and was dried in vacuo at 40° C. during 48 h providing 112.65 g of cis-4-{3-[4-(1,1-dimethyl-propyl)-phenyl]-2-methyl-propyl}-2,6-dimethyl-morpholine hydrochloride as white powder. (64% yield) (Melting Point=205.7° C.).

¹H NMR (400 MHz, CDCl₃) δ: 0.63 (3H, t, J=7.39 Hz), 1.12 (3H, d, J=6.30 Hz), 1.13 (3H, d, J=6.32 Hz), 1.24 (6H, s), 1.26 (3H, d, J=6.65 Hz), 1.59 (2H, q, J=7.49 Hz), 2.05 (1H, q, J=10.77 Hz), 2.26 (1H, q, J=10.80 Hz), 2.27-2.35 (1H, m), 2.56-2.66 (2H, m), 2.76-2.89 (2H, m), 3.22 (2H, t, J=13.28 Hz), 4.36-4.44 (1H, m), 4.46-4.54 (1H, m), 7.06 (2H, d, J=8.22 Hz), 7.23 (2H, d, J=8.22 Hz), 12.55 (1H, b).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A process for the synthesis of 3-[4-(1,1-dimethyl-propyl)-phenyl]-2-methyl-propionaldehyde of formula (II): 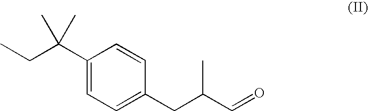
comprising Heck reacting a compound of general formula (VI): 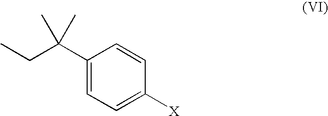
in which X is a halide or a trifluoromethane sulfonate radical (OSO₂CF₃) with 2-methyl-prop-2-en-1-ol of formula (VI): 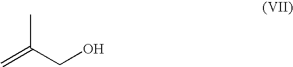
in the presence of a palladium catalyst and a base, in a solvent selected from among N,N-dimethylformamide, polar protic and non-polar organic solvents, and at a temperature ranging from 60° C. to 150° C.

2. A process for the synthesis of Amorolfine of formula (I): 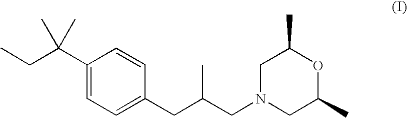
or salt thereof, comprising, in a first step, preparing 3-[4-(1,1-dimethyl-propyl)-phenyl]-2-methyl-propionaldehyde of formula (II): 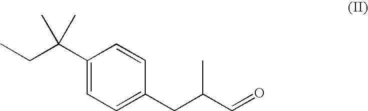
by Heck reacting a compound of general formula (VI): 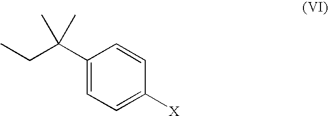
in which X is a halide or a trifluoromethane sulfonate radical (OSO₂CF₃) with 2-methyl-prop-2-en-1-ol of formula (VII): 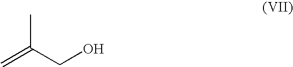
in the presence of a palladium catalyst and a base, in a solvent selected from among N,N-dimethylformamide, polar protic and non-polar organic solvents, and at a temperature ranging from 60° C. to 150° C., and then, in a second step, conducting an amino reduction by reacting said 3-[4-(1,1-dimethyl-propyl)-phenyl]-2-methyl-propionaldehyde of formula (II) with cis-2,6-dimethyl morpholine of formula (IV): 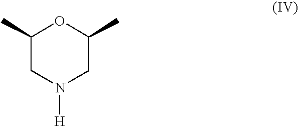
in the presence of a reducing agent and in a solvent.

3. The process as defined by claim 2, comprising converting said Amorolfine into a salt thereof by reacting same with an acid.

4. The process as defined by claim 1, wherein the palladium catalyst employed in the Heck reaction is selected from the group consisting of palladium(II)acetate, palladium(II)chloride, tetrakis(triphenylphosphine)-palladium(0), palladium on activated carbon, dichloro[1,1′-bis(diphenylphosphino)ferrocene]palladium(II) and dichloro-bis-triphenylphosphino palladium(II).

5. The process as defined by claim 1, wherein a phosphinic ligand is employed in combination with the palladium catalyst.

6. The process as defined by claim 5, wherein the phosphinic ligand is selected from the group consisting of triphenylphosphine, tri-o-tolylphosphine, tri-m-tolylphosphine and tri-p-tolylphosphine.

7. The process as defined by claim 1, wherein the Heck reaction is carried out in a polar protic solvent selected from the group consisting of methanol, ethanol, n-propanol, i-propanol, n-butanol, i-butanol, t-butanol and mixture of these solvents with water.

8. The process as defined by claim 1, wherein the Heck reaction is carried out in a non-polar organic solvent selected from the group consisting of tetrahydrofuran, ethyl acetate, toluene, o-xylene, m-xylene and p-xylene.

9. The process as defined by claim 1, wherein the Heck reaction is carried out in a base selected from the group consisting of a tertiary amine, a metal carbonate, and a metal acetate.

10. The process as defined by claim 2, wherein the amino reduction step is carried out in the presence of a reducing agent selected from the group is carried out in the presence of a reducing agent selected from the group consisting of hydrogen gas in the presence of a palladium catalyst and a mixed metal hydride.

11. The process as defined by claim 10, wherein the amino reduction step is carried out in the presence of a palladium catalyst selected from the group consisting of palladium on activated carbon, palladium on activated carbon in the presence of metal salts or Perlmans catalyst.

12. The process as defined by claim 10, wherein the amino reduction step is carried out in the presence of a mixed metal hydride selected from the group consisting of sodium borohydride, lithium borohydride, sodium cyano borohydride and lithium cyanoborohydride.

13. The process as defined by claim 2, wherein the amino reduction step is carried out in a polar protic solvent selected from the group consisting of methanol, ethanol, propanol, i-propanol, butanol, iso-butanol and tert-butanol.

14. The process as defined by claim 2, wherein the amino reduction step is carried out in a non-polar solvent selected from the group consisting of toluene, tetrahydrofuran and ethyl acetate.

15. The process as defined by claim 11, wherein the temperature in the amino reduction step is no more than 45° C.

16. The process as defined by claim 12, wherein the temperature in the amino reduction step ranges from 0° C. to 30° C.

17. The process as defined by claim 2, comprising carrying out said two steps in a one pot process without isolation of the aldehyde intermediate of formula (II).

18. The process as defined by claim 17, comprising the following steps:
a) first reacting (1,1-dimethyl-propyl)-4-iodo-benzene with 2-methyl-prop-2-en-1-ol in N,N-dimethylformamide and in the presence of palladium acetate and sodium bicarbonate, at 100° C. over 9 h;
b) filtering the catalyst through Celite, then adding ethanol, acetic acid and cis-2,6-dimethylmorpholine;
c) cooling the mixture to −5° C., then adding sodium borohydride
d) after work-up, dissolving the crude product obtained from step c) in diisopropyl oxide and adding a solution of hydrochloric acid gas in ethyl acetate;
e) isolating cis-4-{3-[4-(1,1-dimethyl-propyl)-phenyl]-2-methyl-propyl}-2,6-dimethyl-morpholine hydrochloride by filtration.

19. A process for the synthesis of (1,1-dimethyl-propyl)-4-iodo-benzene of formula (VIa): 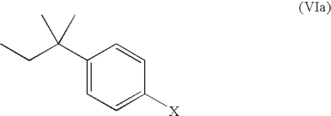
comprising reacting (1,1-dimethyl-propyl)-benzene of formula (VIII): 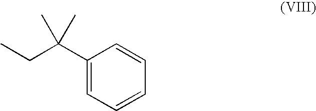
with sodium metaperiodate and iodine in a mixture of acetic acid and acetic anhydride and then adding sulfuric acid to the medium of reaction.
